(19) United States
(12) Patent Application Publication
(10) Pub. No.: US 2024/0277244 A1
(43) Pub. Date: Aug. 22, 2024

(54) PROBE FOR IDENTIFICATION OF OCULAR TISSUES DURING SURGERY

(71) Applicants: Sahba Aghajani PEDRAM, Oakland, CA (US); Tsu-Chin TSAO, Oakland, CA (US); The Regents of the University of California, Oakland, CA (US)

(72) Inventors: Peter Walker FERGUSON, Los Angeles, CA (US); Sahba Aghajani PEDRAM, Los Angeles, CA (US); Matthew GERBER, Los Angeles, CA (US); Jacob Rosen, Los Angeles, CA (US); Jean-Pierre HUBSCHMAN, Los Angeles, CA (US); Tsu-Chin TSAO, Los Angeles, CA (US); Ismael CHEHAIBOU, Los Angeles, CA (US); Anibal FRANCONE, Los Angeles, CA (US)

(73) Assignee: The Regents of the University of California, Oakland, CA (US)

(21) Appl. No.: 18/569,976

(22) PCT Filed: Jun. 14, 2022
(86) PCT No.: PCT/US2022/033484

§ 371 Date: Dec. 13, 2023

Related U.S. Application Data
(60) Provisional application No. 63/210,256, filed on Jun. 14, 2021.

(51) Int. Cl. A61B 5/0537; A61B 5/0538; A61F 9/007

(52) U.S. Cl. CPC A61B 5/0537; A61B 5/0538; A61F 9/00736

(57) ABSTRACT

According to certain general aspects, the present embodiments relate generally to identifying tissue, fluid and/or anatomical structures at the tip of a surgical tool. The determination of the tissue, fluid and/or anatomical structures that the tool is touching allows the inference of a position inside of a person undergoing surgery. For example, a surgeon may attempt to use a tool to interact with a lens portion of a person's eye during cataract surgery, but the identification of tissue provided by embodiments will indicate that the tool is at a position too deep inside of the eye.

### CROSS-REFERENCE TO RELATED APPLICATIONS

The present application is based on and claims priority to U.S. Provisional Patent Application No. 63/210,256 filed Jun. 14, 2021, the contents of which are incorporated herein by reference in their entirety.

### STATEMENT OF GOVERNMENT RIGHTS

This invention was made with government support under Grant Number EY024065, awarded by the National Institutes of Health. The government has certain rights in the invention.

### TECHNICAL FIELD

The present embodiments relate generally to tissue and fluid identification, and more particularly to identifying ocular tissues during cataract surgery.

### BACKGROUND

Cataracts are very common and cause a person's eye lens to get cloudy, thereby obscuring vision. This because the lens is the part of the eye that is responsible for focusing light necessary to create clear images of objects at various distances. The lens is located inside the capsular bag, which is behind the iris and the cornea. The capsular bag is very delicate and translucent. Cataract surgery to treat cataracts is also very common. During cataract surgery, an incision is made in the cornea and the cataract may either be removed in its entirety, or broken up via an ultrasonic probe or a laser. After removal, the lens is replaced with an artificial lens.

Cataract surgery includes many manual steps, which are thus prone to human error and are time consuming. For example, the broken pieces of the lens must manually be identified and removed via suction or irrigation and aspiration. In some circumstances, lens material can accidently remain in the capsular bag. Surgeons performing cataract surgery may believe they have cleared the capsular bag of all lens material, unknowingly leaving lens material behind for example, the iris, because the iris blocks the surgeon's complete view of the capsular bag. There is no known imaging technology able to penetrate the opaque iris such that the surgeon can see through the iris and into the capsular bag. Completely removing the lens pieces of the eye reduces the likelihood of secondary cataracts. Secondary cataracts may form after a person has undergone cataract surgery and impair a person's vision.

There are many reasons why surgeons performing cataract surgery may have limited visual feedback. For example, the surgeon's tool or hand may prohibit the surgeon from completely visualizing the eye. Conventionally, surgeons can use microscopes in an attempt to enhance their visual field. However, side-by-side display of the information provided from the microscopes to the surgeons during surgery can increase the difficulty of the surgery. For example, a surgeon cannot look at the microscope images without first taking their own eyes off of their workspace.

In other attempted solutions at improving visual feedback during surgery, information indicating the position of the tool inside of the human eye is provided via Ocular Coherence Tomography (“OCT”). OCT can provide depth information of the eye such that the position of the tool inside can be determined. However, the time required to scan the eye and perform depth analysis can take several seconds, whereas the normal human reaction time is approximately 250 ms. Thus, a determination that a surgical tool is in an undesirable location in the eye cannot be corrected by a surgeon quickly enough in real time using OCT.

Therefore, many obstacles remain in the goal of automating the cataract surgery process, for example in determining a tool's position in the eye without direct visualization. It is against this technological backdrop that a technological solution to these and other problems rooted in this technology was sought by the present Applicant.

### SUMMARY

According to certain general aspects, the present embodiments relate generally to identifying tissue, fluid and/or anatomical structures at the tip of a surgical tool. The determination of the tissue, fluid and/or anatomical structures that the tool is touching allows the inference of a position inside of a person undergoing surgery. For example, a surgeon may attempt to use a tool to interact with a lens portion of a person's eye during cataract surgery, but the identification of tissue provided by embodiments will indicate that the tool is at a position too deep inside of the eye. Armed with this and other information, the present embodiments enable the surgeon to take corrective and/or preemptive actions.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIG. 1 is a diagram of the side view of the anatomy of an eye.

FIGS. 2A-2C are diagrams of an example embodiment of a tool used to detect the type of tissue, fluid and/or anatomical structures at the tip of the tool, according to embodiments.

FIG. 3 is a functional block diagram of an example system used for detecting the tissue at the tip of a tool, according to embodiments.

FIG. 4A is a diagram of an example of the electric circuit, according to embodiments.

FIG. 4B illustrates an example input signal and an example output signal response given the input signal according to embodiments.

FIGS. 5A-5C are diagrams of the input-output voltages of various eye tissues at various frequencies and for various numbers of samples of tissues taken at various times, including responses in both magnitude and phase.

FIG. 6 is a flowchart illustrating an example method of classifying tissue, fluid and/or anatomical structures based on impedance, according to embodiments.

FIG. 7A illustrates confusion matrices of several classification algorithms with respect to the algorithms' reliability.

FIG. 7B illustrates confusion matrices of several classification algorithms with respect to the algorithms' sensitivity.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

According to certain aspects, the present embodiments are related to identifying tissue, fluid and/or anatomical structures at the tip of a tool and determining the position of the tool within a body. While tissue, fluid and/or anatomical structures are described, tissue, fluid and/or anatomical structures may include, but are not limited to, lens material such as the nucleus, cortical material, and capsular bag, cornea tissue, iris tissue, vitreous bodies, retina layers such as the internal limiting membrane (“ILM”), retinal pigment epithelium (“RPE”), and photoreceptors, ciliary bodies, epiretinal membranes, blood, viscoelastic gel, balanced salt solution (“BSS”), and distilled water. Further, while cataract surgery is described, tools used to perform other surgeries can be modified such that the tool can identify tissue, fluid and/or anatomical structures in real-time during the surgery, after those skilled in the art have been taught by the present examples. Additional details and explanation of the various techniques and uses described herein by be appreciated with reference to Pedram et al., “A Novel Tissue Identification Framework in Cataract Surgery using an Integrated Bioimpedance-Based Probe and Machine Learning Algorithms,” by Pedram et al., IEEE Transactions on Biomedical Engineering (2021), incorporated herein by reference in its entirety.

Among other things, the present Applicant recognizes that the anatomy of a human eye makes determining the tissue/fluid/anatomical structure in contact with a tool inside of an eye difficult because surgeons performing cataract surgery may not have direct visualization of a tip of a tool inside of an eye.

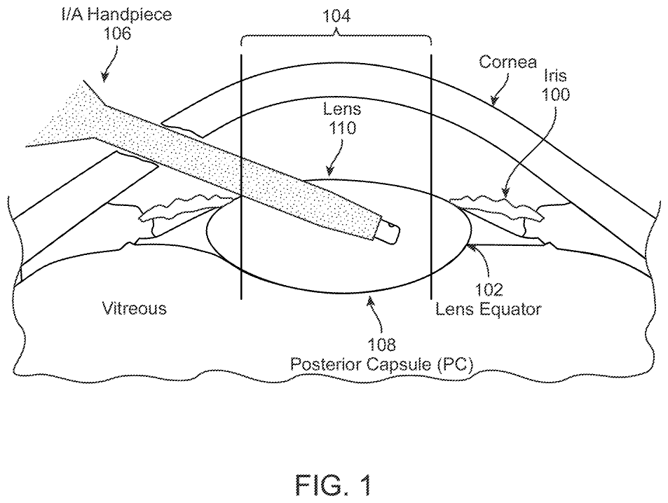

In this regard, FIG. 1 is a diagram of the side view of the anatomy of an eye. The iris 100 blocks most of a region of the eye called the lens equator 102 from vision. The area between the lines 104 is what is visible to the surgeon, from a side view. In addition, during surgery, the irrigation/aspiration hand piece 106 further reduces a surgeon's view of the posterior capsule 108 and lens 110. This figure helps illustrate one problem recognized by the present Applicant of the surgeon's reduced field of view of the eye.

According to certain general aspects, therefore, the present embodiments aim to remedy this and other problems by allowing a user to determine the tissue, fluid and/or anatomical structures that the tip of their tool touches, including but not limited to lens material such as the nucleus, cortical material, and capsular bag, cornea tissue, iris tissue, vitreous bodies, retina layers such as the internal limiting membrane (“ILM”), retinal pigment epithelium (“RPE”), and photoreceptors, ciliary bodies, epiretinal membranes, blood, viscoelastic gel, balanced salt solution (“BSS”), and distilled water, without a dependency on visualizing the tissue, fluid and/or anatomical structures during a surgery.

In embodiments, a tool in accordance with these and other aspects comprises two conductors that are insulated from each other except at their distal ends. At the distal end of the tool, the two conductors can align with the tip of the tool, remaining separate from each other. In some example embodiments, conductors may be 18 gauge copper wire or steel needle. The conductors can be routed through the interior or exterior of the tool such that they do not modify the geometry of the tool. Similarly, the routing of the conductors may be achieved such that the conductors do not affect the performance of the tool in its function. In some embodiments, the tool itself can serve as one or both of the conductors. The conductors can be electrically coupled to a circuit.

While a probe used in cataract surgery is discussed herein, the concepts applied to the probe can be integrated into other tools by those skilled in the art after being taught by the present examples. Specifically, the embodiments herein can be applied to other intraocular tools, including but not limited to irrigation/aspiration hand pieces, vitreous cutters, and intraocular forceps, as will be appreciated by those skilled in the art. In other embodiments, the probe is a standalone unit that is separate from other surgical tools.

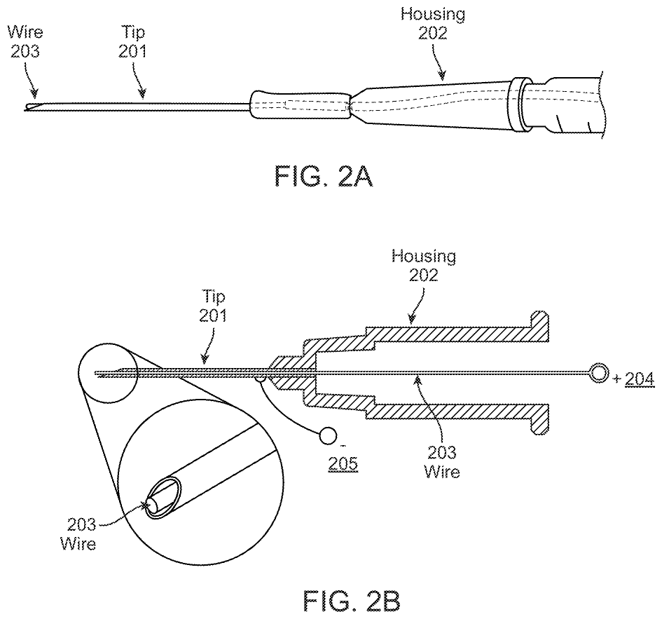

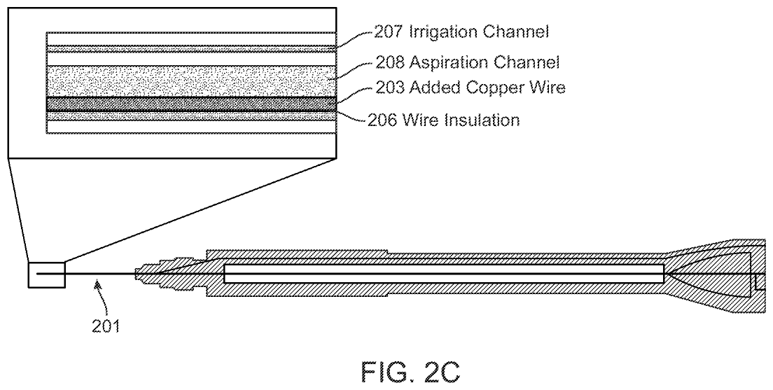

In accordance with these and other aspects, FIG. 2A is a diagram of an example embodiment of a tool used to detect the type of tissue, fluid and/or anatomical structures at the tip of the tool, according to embodiments. The tool can comprise a tip of the tool 201 inside of housing 202. In some embodiments, the tip of the tool 201 can be hollow such that a wire 203 can be inserted inside of the tip of the tool 201. The wire 203 inside of the tip of the tool 201 can comprise one path of an electric circuit, while another path of the same electric circuit can be the tip of the tool 201 itself.

FIG. 2B is another diagram of an example embodiment of a tool used to detect the type of tissue, fluid and/or anatomical structures at the tip of the tool, according to embodiments. The hollow tip of the tool 201 may hold the wire 203 in the housing 202. The wire 203 may comprise one path of an electric circuit 203. The tip of the tool 201, insulated from the wire 203, may comprise the second path of the same electric circuit 205. The electric circuit can be completed when the wire 203 inside of the tip of the tool touches another conductive material, for example tissue, fluid and/or anatomical structures. In some embodiments, less than one millimeter of wire 203 may be exposed at the tip of the tool 201.

FIG. 2C is another diagram of an example embodiment of a tool used to detect the type of tissue, fluid and/or anatomical structures at the tip of the tool, according to embodiments. As discussed herein, a tool can be constructed such that two conductors may be insulated from each other except at their distal ends. The wire insulation 206 insulates one conductor, the copper wire 203, from the other conductor, the tip of the tool 201. The wire 203 and insulation 206 can be routed through the interior of the tool such that they do not modify the geometry of the tool and/or affect the performance of the tool. As shown in the example of FIG. 2C, the probe, with irrigation and aspiration functionalities, continues to provide irrigation and aspiration functions via the irrigation channel 207 and the aspiration channel 208, respectively, which are not disturbed by the copper wire 203 and insulation 206. This illustrates an aspect of the embodiments, which is that the probe to identity types of tissue, fluid and/or anatomical structures can be easily integrated into a surgical tool such that a surgeon using the tool can have access to information (e.g. real time) provided by the probe according to embodiments.

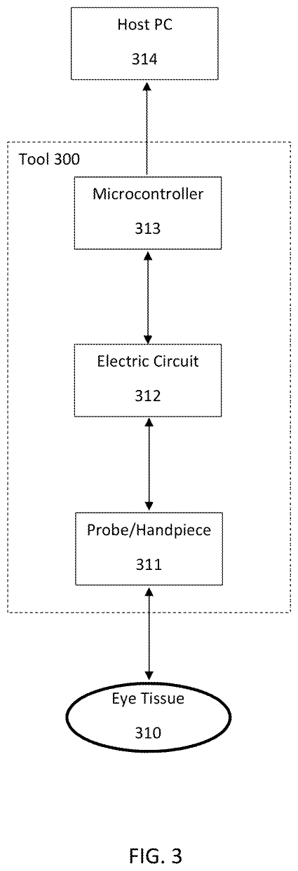

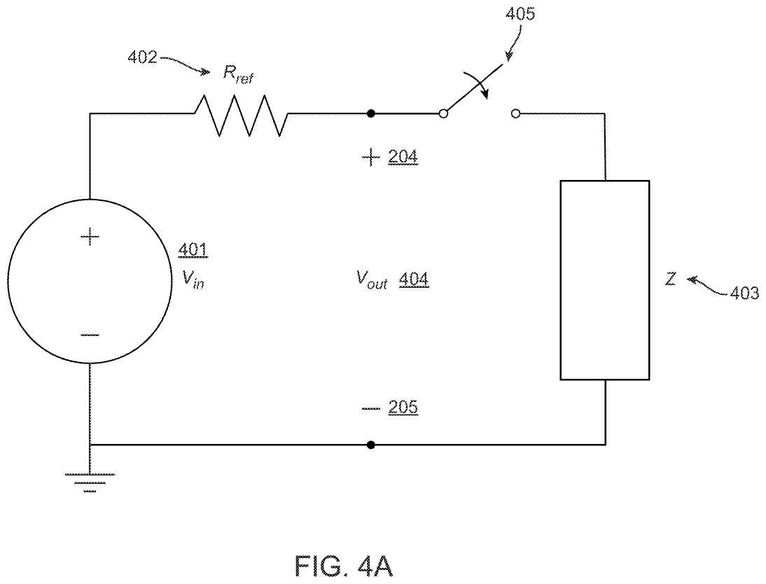

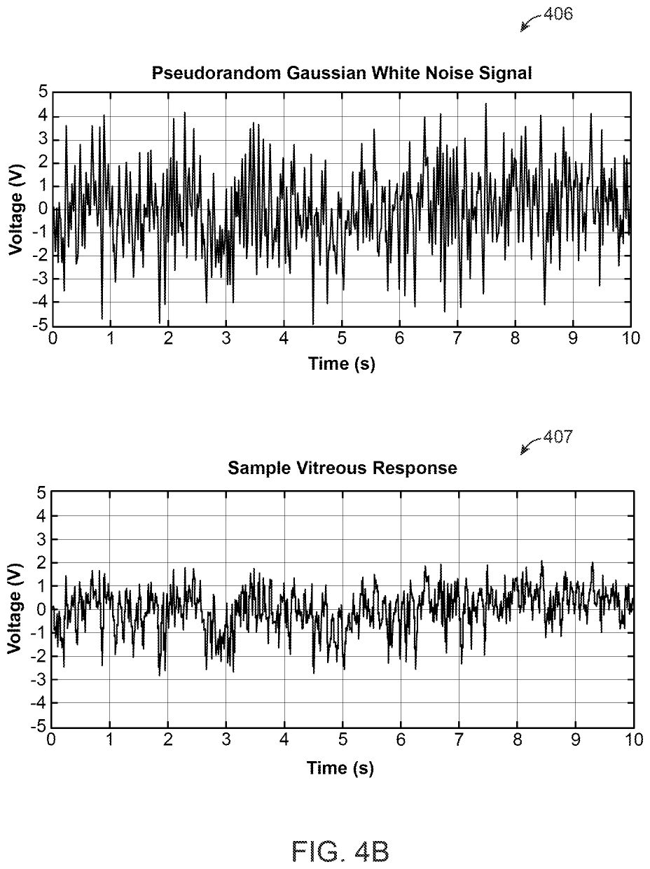

As set forth above, the tip 201 of the tool can touch tissue, fluid and/or anatomical structures such that the tissue, fluid and/or anatomical structures completes an electric circuit and an electrical signal travels through the tissue, fluid and/or anatomical structure, thereby detecting contact between the tool and the tissue, fluid and/or anatomical structure. In response to the completed circuit and/or detected contact, a voltage will be applied to the tissue, fluid and/or anatomical structures such that a response of the tissue, fluid and/or anatomical structures can be determined via the tool and the electric circuit. The electric circuit can be any circuit where the impedance of a load can be calculated. For example, the electric circuit can be a voltage divider circuit or a Wheatstone bridge. A diagram of an example electric circuit is illustrated and will be described below in connection with FIGS. 3 and 4 herein.

A processor in or coupled to the tool can determine the impedance of the tissue, fluid and/or anatomical structures based on the measured response at the completed electric circuit caused by the tip 201 of the tool touching the tissue, fluid and/or anatomical structures. Further, the processor can be used to classify the tissue, fluid and/or anatomical structures based on the determined impedance. In some embodiments, a processor can be used to determine the impedance and classify the tissue, fluid and/or anatomical structures. In other embodiments, a data acquisition device such as a microcontroller can be used to determine the impedance, while a different device such as a computer with a processor can be used to classify the tissue, fluid and/or anatomical structures.

Artificial intelligence can be implemented in the processor to classify the tissue, fluid and/or anatomical structures and provide the classification to a user. Artificially intelligent systems can include, but are not limited to support vector machines (“SVM”), AdaBoost, Decision Trees, Convolutional Neural Networks, Decision Trees, Random Forests, and Stochastic Gradient Descent algorithms.

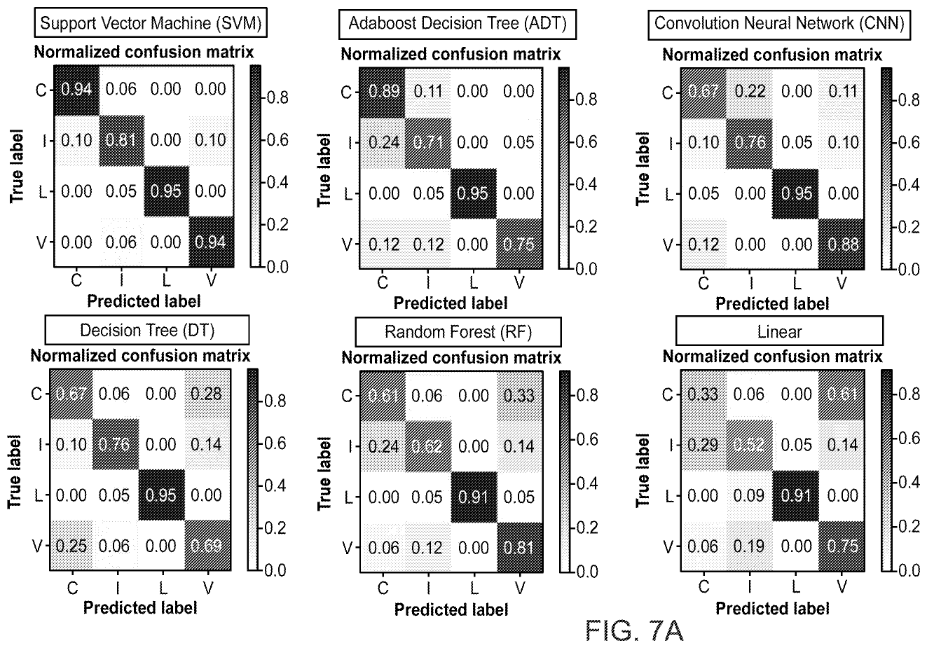

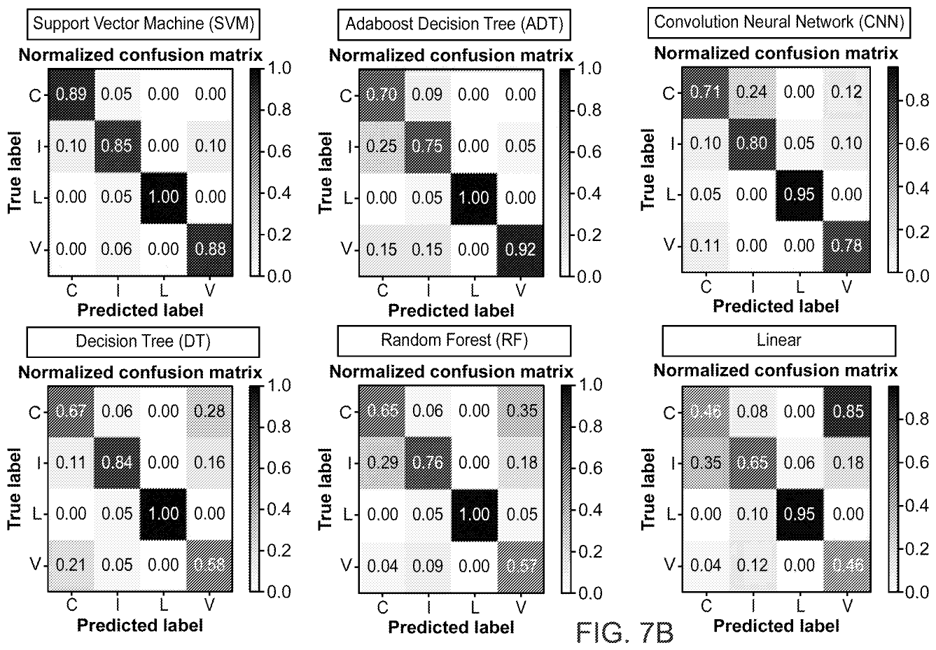

In some embodiments, the SVM algorithms can be implemented because testing indicated that SVMs classified tissues with the highest reliability, sensitivity, and average, as compared to other artificial intelligence algorithms. FIGS. 7A-7B indicate that in some environments SVMs achieve the best classification results as compared to AdaBoost Decision Trees, Convolutional Neural Networks, Decision Trees, Random Forests, and Stochastic Gradient Descent algorithms, which can be considered a type of linear regression model.

FIG. 7A illustrates confusion matrices of several classification algorithms with respect to the algorithms' reliability. The reliability of the classification describes the likelihood of the classifier's correct classification. In other words, when the classifier predicted that the tissue was a particular class of tissue, the reliability assesses the likelihood that the predicted class is the actual class. The reliability of the classification algorithm can be expressed by Equation 1 below.

$$Reliability = {C}_{R} = \frac{\vert y∩ \hat{y} \vert}{\vert \hat{y} \vert} \qquad Equation 1$$

In Equation 1 above, y represents the set of true labels and ŷ represents the set of predicted labels. As is commonly indicated, ∩ represents the intersection of the two labels.

The tissues classified were the cornea (“C”), iris (“I”), lens (“L”) and vitreous material (“V”). The tissue classes are on the x and y-axis of the matrix, where the x-axis indicates the predicted labels and the y-axis indicates the true labels. When evaluating confusion matrices, the diagonal values are important because the predicted label is the same as the true label. In other words, a 1.0 in a diagonal cell would indicate that the classifier predicts the actual class 100% of the time. The columns of reliability confusion matrices indicate the likelihood of the other tissue classification. For example, through analysis of the first column of the first confusion matrix, it can be shown that the SVM predicted the cornea tissue with 89% accuracy. If the SVM didn't classify the cornea tissue as cornea tissue, the SVM classified the cornea tissue as iris tissue 10% of the time. Thus, the classifier with the largest values across the diagonal of the matrix performs the best. As indicated in FIG. 7A, the SVM produced the most reliable classifications.

FIG. 7B illustrates confusion matrices of several classification algorithms with respect to the algorithms' sensitivity. The sensitivity of the classification describes the likelihood of the algorithm detecting a particular class. In other words, when the tool touches a specific tissue, the sensitivity assess the probability that the classifier can determine that tissue. The sensitivity of the classification algorithm can be expressed by Equation 2 below.

$$Sensitivity = {C}_{S} = \frac{\vert y∩ \hat{y} \vert}{\vert y \vert} \qquad Equation 2$$

In Equation 2 above, y represents the set of true labels and § represents the set of predicted labels. As is commonly indicated, n represents the intersection of the two labels.

The tissues classified were the cornea (“C”), iris (“I”), lens (“L”) and vitreous material (“V”). The tissue classes are on the x and y-axis of the matrix, where the x-axis indicates the predicted labels and the y-axis indicates the true labels. When evaluating confusion matrices, the diagonal values are important because the predicted label is the same as the true label. In other words, a 1.0 in a diagonal cell would indicate that the classifier predicts the actual class 100% of the time. The rows of sensitivity confusion matrices indicate the likelihood of the other tissue classification. For example, through analysis of the first row of the first confusion matrix, it is clear that the SVM predicted the cornea tissue with 89% accuracy. If the SVM didn't determine that the probe was touching the cornea tissue, the SVM predicted that the probe was touching iris tissue 5% of the time. Thus, the classifier with the largest values across the diagonal of the matrix performs the best. As indicated in FIG. 7B, the SVM produced the most sensitive classifications.

The accuracy of the classification algorithms, or the general performance of the algorithms, can be determined by averaging the reliability and sensitivity ratings. The accuracy of the classification algorithm can be expressed by Equation 3 below.

$$Accuracy \frac{2}{{C}_{S}^{- 1} + {C}_{R}^{- 1}} = \frac{2 {C}_{S} {C}_{R}}{{C}_{S} + {C}_{R}} \qquad Equation 3$$

Table 1 below illustrates the results of the accuracy analysis.

TABLE 1

| Algorithm | Accuracy |
|---|---|
| SVM | 0.91 |
| AdaBoost Decision Tree | 0.84 |
| Convolutional Neural Network | 0.81 |
| Decision Tree | 0.77 |
| Random Forest | 0.74 |
| Stochastic Gradient Descent | 0.63 |

As illustrated in Table 1 above, SVMs classified the eye tissue more accurately than the other classifiers, given the impedances of the eye tissue.

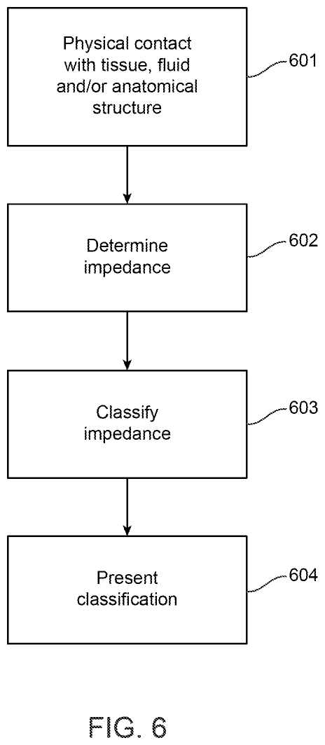

The SVM algorithm is a means of classification by finding an ideal line or hyperplane between multiple classes of data. In the present embodiment, the impedance of various eye tissues are distinguishable enough such that the tissue can be classified given the impedance. In other words, the input to the SVM can be an impedance value, and the output is a tissue, fluid and/or anatomical structures classification for the input impedance. FIGS. 6A-6C discussed herein are diagrams of the impedances of various eye tissues at various frequencies.

The SVM can classify data by determining the ideal line or hyperplane between the data. For example, given two classes of data represented by data points on a graph, the SVM will attempt to find a hyperplane that distinguishes the classes of data. During training, in a supervised model, the classes of data associated with the various data points are known. Artificially intelligent systems may be trained on known input/output pairs such that the artificial intelligence can learn how to classify an output given a certain input. In the present embodiment, an input/output pair can be an impedance value and a tissue classification. Once the artificial intelligence has learned how to classify known input/output pairs, the artificial intelligence can operate on unknown inputs to predict what the classified output should be.

The more diverse the sample set is, the more robust the artificially intelligent system can be in its classifications. For example, an artificially intelligent system will attempt to classify input/output pairs during a first iteration of learning. If, during a next iteration of learning, the input/output pairs are similar to the learned input/output pair of the first iteration, the artificially intelligent system may coincidentally perform higher than it should perform merely because the data is similar, and not because the artificially intelligent system is robust. If a diverse input/output pair is subsequently input to the artificially intelligent system for the third iteration, the classification error will likely be much higher than it would be if the first two input/output pairs were diverse. The similarity of the first two input/output pairs might cause the artificially intelligent system to fine tune itself to the similar input/output pairs of the first two iterations. This may be called “overtraining” the system. In the context of SVMs, the separating boundary between the classes can be considered too close to the data such that the separating boundary is not general enough to classify diverse data.

Alternatively, if the second iteration of training used a distinct input/output pair compared to the input/output pair of the first iteration, the artificially intelligent system would be forced to be able to classify a broader range of input/output pairs because the separating boundary would need to be more drastically tuned such that it learns the new input/output pair. During testing, the outputs are not known so it is ideal for the artificially intelligent system to be able to classify a broad range of input/output pairs

For a SVM, given a set of data points, a separating boundary can be determined that classifies the data and the equation of the boundary can be stored in memory. Given a new batch of input/output pairs, the equation of the boundary stored in memory can be used in an attempt to classify the new data. The equation of the boundary can be tuned such that it fits the new batch of input/output pairs more ideally. The artificially intelligent system changes over time because the classification boundary is tuned as more input/output pairs are learned.

The SVM will consider various data points and the distances between the points until the SVM determines the closest pair of data points that are in different classes. These data points can be considered support vectors. The SVM will subsequently determine the equation of a plane between the support vectors, creating a boundary between the separate classes. The distance between the support vectors of each class and the boundary are maximized such that the maximum amount of space exists between the boundary separating the classes and the support vectors. Data points closest to the boundary have a higher likelihood of being misclassified. Thus, the more space between the separating boundary and the data can mean that the separating line is more generalized, creating a more robust classification scheme.

In some embodiments, if the data is nonlinear, the dimension of the data can be increased such that a plane that distinguishes the classes of data can be determined. Subsequently, the data and the equation of the separating plane are converted back to the original dimension. The conversion of the data and equation of the separating plane to different dimensions can be performed using known methods, for example, by increasing the number of features in the data set. In alternate embodiments, if the data is nonlinear, a kernel function can be applied to the data to evaluate the similarity of the data such that distances of the data can be approximated without having to determine the actual distance of data in a higher dimensional space.

In some embodiments, the SVM can be trained via the manual mapping of impedance values to a class. For example, an impedance can be measured and a user can label the type of tissue, fluid and/or anatomical structures associated with the impedance. In other embodiments, the SVM can be trained via databases of impedance values that have been mapped to known tissue, fluid and/or anatomical structures.

During testing, the SVM uses the tuned equation learned during the training phase. An impedance can be determined via a processor in response to the tip of the tool touching a conductive surface and completing the electric circuit. The impedance can be classified by the SVM such that the class of the tissue touching the tool can be determined.

FIG. 3 is a diagram providing an example overview of a system used for detecting the tissue at the tip of a tool 300, according to embodiments. The probe 311 (e.g. including the tip, wire and housing components shown in the examples of FIGS. 2A to 2C) physically touches the eye tissue 310 such that the electric circuit 312 is completed. The electric circuit 312 that is electrically coupled to the probe 311 provides the probe with an input voltage (e.g. an AC voltage with a specific single, complex or variable frequency). A microcontroller 313, such as the microcontroller myRIO manufactured by National Instruments, can provide the input signal to the electric circuit 312, or can otherwise control a voltage source in electric circuit 312. When the circuit is completed, an output voltage can be measured via the probe 311 at the electric circuit 312.

The electric circuit 312 can provide the response of the completed circuit via analog signals back to the microcontroller 313 (e.g. via an analog-to-digital converter ADC and/or filters, not shown). The microcontroller can perform circuit analysis based on the received analog signals to determine the impedance of the eye tissue 310. The microcontroller 313 can be electrically coupled to a host PC 314 such that the host PC 314 can perform the tissue classification. In some embodiments, the microcontroller 313 is electrically coupled to the host PC 314 via a Universal Serial Bus (“USB”) connection or any other suitable wired or wireless (e.g. Bluetooth) connection. The microcontroller 313 may provide the host PC 314 digital signals such that a processor in the host PC 314 can perform tissue classification via an artificially intelligent system (e.g. using SVMs as described above). In some other embodiments, microcontroller 313 and/or other processors within tool 300 can perform tissue classification.

It should be noted that tool 300 can include other components for performing surgery, such as the components shown in the example of FIG. 2C.

FIG. 4A is a diagram of an example of the electric circuit that can be used to implement circuit 312 according to embodiments. A voltage V_{IN} 401 can be applied to a circuit. For example, the voltage V_{IN} can be a signal ranging from ±5V. V_{IN} 401 can be any input signal including, but not limited to DC step, chirp or impulse signals or AC signals such as sinusoidal sweeps, pseudorandom white noise or a single AC frequency voltage (e.g. 1 kHz). The voltage V_{IN} can be generated via any method and corresponding electrical component of generating a voltage signal as will be appreciated by those skilled in the art.

In some embodiments, pseudorandom white noise is used as an input signal because the white noise characteristics can be applied to the circuit consistently and quickly each time V_{IN} 401 is applied to the circuit. A known resistor, R_{REF} 402 can be used to determine the impedance, as discussed further herein and as appreciated by those skilled in the art. Referring to FIGS. 2A and 2B, a positive path of a circuit 204 can be one portion of a circuit, while a negative path of the circuit 205 can be a second portion of a circuit. For example, the positive path 204 can be conducted via wire 203 while the tip of the tool 201 can conduct the negative path 205 of the circuit.

As is commonly understood, the circuit will not be completed and thus no current will flow through the circuit if a path of the circuit is open. The switch 405 indicates that the circuit remains in an open state until the circuit is proactively closed. The circuit can become closed when the wire 203 and tip of the tool 201 touch a conductive material. When the wire 203 and tip of the tool 201 touch a conductive material, the switch 405 is effectively closed and electricity can flow through the circuit. It should thus be appreciated that switch 405 is shown for illustration, and may not be actually implemented using a dedicated electrical component. An output voltage V_{OUT} 404 can be measured across the conductive material. Similarly, the impedance of the conductive material Z 403 can be calculated using well known circuit analysis as shown in Equation 4.

$$\begin{matrix}{V}_{O U T} = {V}_{IN} \times ( \frac{Z}{Z + {R}_{R E F}} ) \\ Z = \frac{{V}_{O U T} ( 1 + {R}_{R E F} )}{{V}_{IN}}\end{matrix} \qquad Equation 4$$

In some embodiments, a low pass filter may be placed in the circuit to filter out unwanted frequencies. For example, in determining the impedance of various eye tissues, it was determined that frequencies over about 20 Hz tend to not generate useful information. Thus, a 20 Hz low pass filter can be implemented to filter out the higher frequencies.

FIG. 4B illustrates an example response given an input signal. Signal 406 illustrates an input signal where the input is a pseudorandom white noise signal from ±5V. The x-axis describes the signal over time, in seconds, while the y-axis describes the voltage range. Signal 407 is the response produced when the tool touches vitreous material. As discussed herein, when the electric circuit is completed, an output voltage can be measured. The output voltage can be used to determine the impedance of the touched tissue. The x-axis describes the signal over time, in seconds, while the y-axis describes the voltage range.

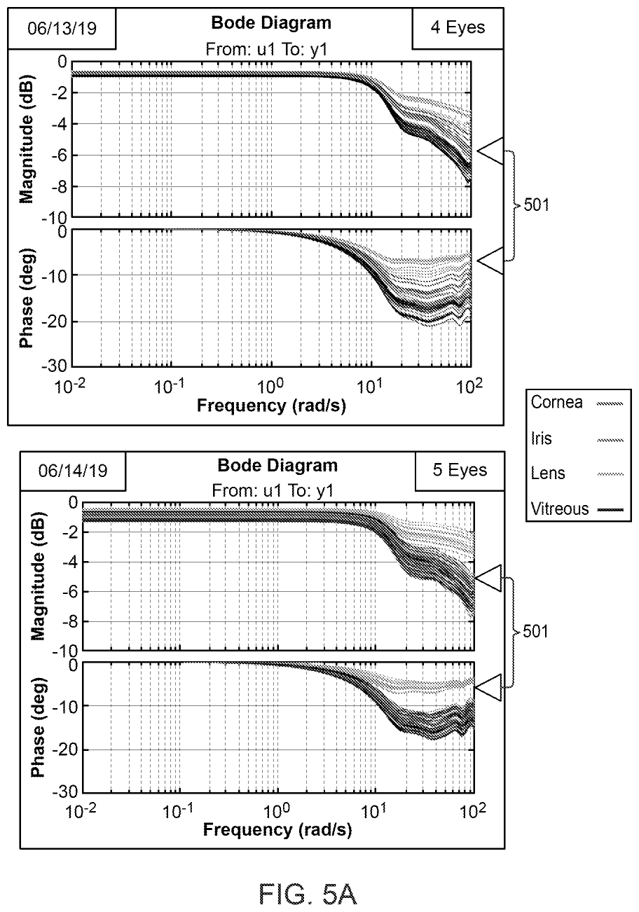

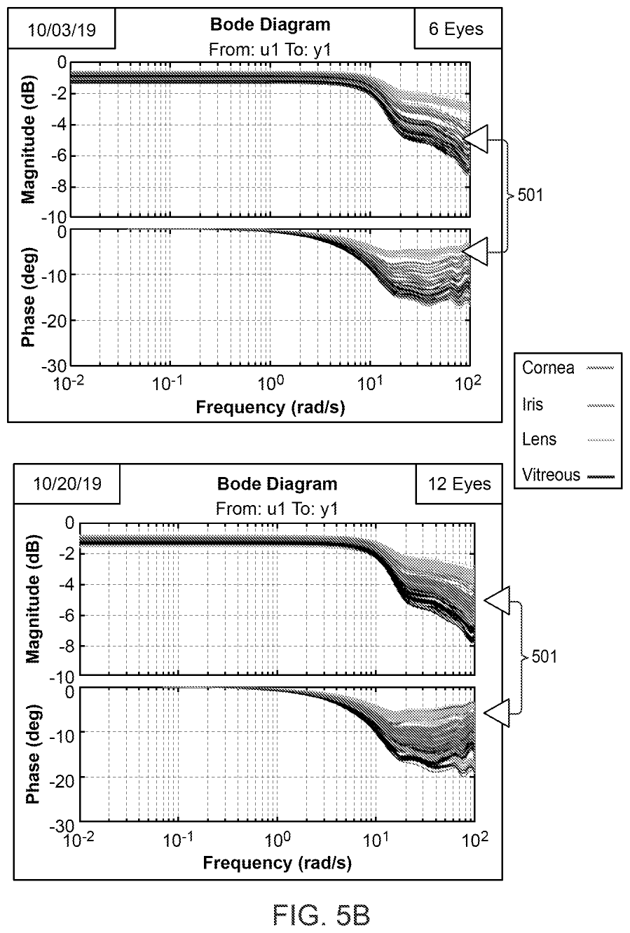

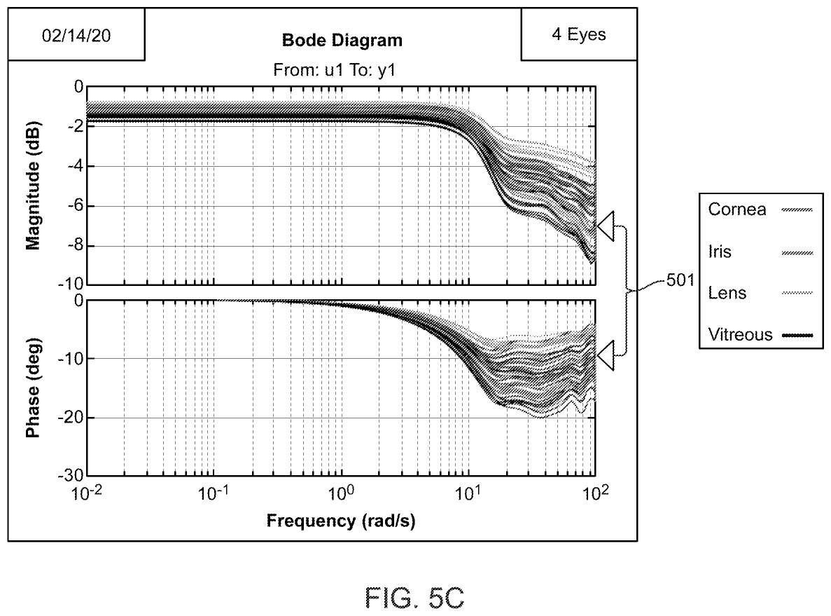

FIGS. 5A-5C are diagrams of the input-output voltages (magnitude and phase) of various eye tissues at various frequencies from DC up to about 100 radians/sec using measurements from different numbers of sample at different times. As discussed herein, the input-output voltages (with respect to the input voltages) can be used to determine the impedance. The measured eye tissue include the cornea, iris, lens, and vitreous material in this example.

The example diagrams in FIGS. 5A-5C are Bode Plots for magnitude and phase of the input-output voltage relationship. A Bode Plot is a plot of the relationship of V_{OUT} to V_{IN} in the frequency domain. For example, a pseudorandom white noise input can trigger various responses of tissue at different frequencies. The Bode Plots illustrated in FIGS. 5A-5C indicate that various eye tissues, for example, cornea, iris, lens and vitreous tissue (represented by different shaded curves, respectively), have significantly different input-output responses 501 in both magnitude and phase at certain frequencies. The impedance (e.g. as a function of frequency or specific frequencies) can be calculated for the distinct input-output responses using the equations above, for example, and used for classification. Further, a classifier can distinguish the impedances and the classification of a tissue, fluid and/or anatomical structures is thus possible through analysis of the impedance.

The example diagrams illustrated in FIGS. 5A-5C further illustrate that many samples of input-output voltages can be obtained using a single or similar tools, for the same or a number of different tissues (e.g. cornea, iris, lens, vitreous tissue) and the results can be stored in a database of known samples. These raw results can be stored in a database, or merely used to train or update the training of a machine learning model (e.g. SVM) that is then used during real time for real-time classification using the same or similar tool and/or the same or similar input voltages.

FIG. 6 is a flowchart illustrating an example method of classifying tissue, fluid and/or anatomical structures based on impedance, according to embodiments.

In block 601, a tool is constructed and/or prepared such that two conductors may be insulated from each other except at their distal ends. The tool can be electrically coupled to a circuit. The tool's physical contact with tissue, fluid and/or anatomical structures can complete the electric circuit such that a response can be measured.

In block 602, based on the measured response from the completed circuit, the impedance of the tissue, fluid and/or anatomical structures can be determined (e.g. as a function of frequency and/or for specific frequencies). In some embodiments, a voltage divider circuit can be used to determine the impedance. The input voltage, output voltage, current, and components in the circuit are all known. Thus, the impedance of the tissue, fluid and/or anatomical structures can be calculated using conventional circuit analysis techniques.

In block 603, the determined impedance (e.g. as a function of frequency or specific frequencies) can be provided to a processor such that an artificially intelligent system can classify the impedance. In some embodiments, a trained SVM can classify a tissue, fluid and/or anatomical structures based on an impedance.

In block 604, the classification of the tissue and/or impedance can be presented to a user. In some embodiments, the classification can be presented to a user visually. For example, the tissue, fluid and/or anatomical structures type can be displayed on a screen. In other embodiments, the classification can be presented to a user audibly. For example, a speaker system can be used to speak the tissue, fluid and/or anatomical structures that the tool is touching.

The presentation of the tissue, fluid and/or anatomical structure classification to the user can be done in real time. In some embodiments, the measurement of the tissue, fluid and/or anatomical structures can be determined in as little as 10 ms. Further, the classification of the tissue, fluid and/or anatomical structures can be very fast. Thus, the user will be informed of the tissue, fluid and/or anatomical structures that is in contact with the tool in real time. For example, a probe according to embodiments is able to provide information that the probe is in contact with “correct tissue” or “expected tissue” and has not deviated or caused damage, such as posterior capsule rupture.

The various implementations of the probe/tool combination described above may be applied in different combinations to enable the advantageous tissue identification, discrimination and classification techniques describe above to be applied in a variety of different surgical instruments such as an irrigation/aspiration (I/A) handpiece, a phacoemulsification probe, an injector for intraocular lens implants, ophthalmic syringes, curved syringes for viscoelastic injection as well as adaptions for other tools employed in therapy, intervention or treatment of disorders of the eye. Advantageously, various embodiments described herein may be recombined to benefit other clinical and surgical environments where different electrical behavior or response is likely, a probe embodiment is incorporated into a surgical implement and the data acquisition and classification is conducted in a framework allowing use by a medical practitioner in a real time setting for the clinical circumstances. Still further, considering other application in ophthalmology, alternative embodiments may find a probe adapted and configured for integration into surgical instruments specific to retinal surgery (e.g. vitreous cutter, a wide range of forceps and scissors, trocars, infusion cannulas, membrane scrapers, illumination/chandelier/light probes, endolasers) with a data acquisition and algorithm applied to discriminate or classify other tissue and structures in the eye, such as, for example, sclera, vitreous, retina (in general), limiting membrane (ILM), choroid, epiretinal membrane. In still other alternative embodiments, aspects of the present invention described herein may advantageously classify and provide feedback in real time for one or more or combinations of a cornea, a lens (nucleus and cortical material), an iris, an anterior capsule (AC), and a posterior capsule (PC).

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A system for identifying a sample within a surgical site, the system comprising:
a probe configured to be integrated into a surgical tool;
a circuit coupled to the probe for obtaining a response signal when the probe contacts the sample; and
a processor for classifying the sample in contact with the probe based on the response signal.

2. The system of claim 1, wherein the surgical tool is used for cataract surgery, and the sample is human eye tissue.

3. The system of claim 2, wherein the human eye tissue is one of a cornea, an iris, a lens or vitreous tissue.

4. The system of claim 1, wherein the response signal represents an impedance of the sample.

5. The system of claim 1, wherein the processor implements a machine learning algorithm for performing the classifying.

6. The system of claim 5, wherein the machine learning algorithm includes SVM.

7. The system of claim 1, wherein the probe is further configured to generate an input signal when the probe contacts the anatomical structure.

8. The system of claim 7, wherein the input signal is an alternating current (AC) voltage signal.

9. The system of claim 8, wherein the AC voltage signal is a pseudorandom white noise signal.

10. The system of claim 7, wherein the processor is configured to generate an impedance for one or more frequencies using the response signal and information regarding the input signal.

11. A method for identifying a sample within a surgical site, the system comprising:
configuring a probe for integration into a surgical tool;
coupling a circuit to the probe for obtaining a response signal when the probe contacts the sample; and
classifying the sample in contact with the probe based on the response signal.

12. The method of claim 11, wherein the surgical tool is used for cataract surgery, and the sample is human eye tissue.

13. The method of claim 12, wherein the human eye tissue is one of a cornea, an iris, a lens or vitreous tissue.

14. The method of claim 11, wherein the response signal represents an impedance of the sample.

15. The method of claim 11, wherein the classifying includes a machine learning algorithm.

16. The method of claim 15, wherein the machine learning algorithm includes SVM.

17. The method of claim 11, further comprising generating an input signal when the probe contacts the anatomical structure.

18. The method of claim 17, wherein the input signal is an alternating current (AC) voltage signal.

19. The method of claim 18, wherein the AC voltage signal is a pseudorandom white noise signal.

20. The method of claim 17, wherein the classifying includes generating an impedance for one or more frequencies using the response signal and information regarding the input signal.
